(19) United States
(12) Patent Application Publication
(10) Pub. No.: US 2026/0086075 A1
(43) Pub. Date: Mar. 26, 2026

(54) METHOD AND STATION FOR MEASURING AIRBORNE MOLECULAR CONTAMINATION

(71) Applicant: PFEIFFER VACUUM, Annecy (FR)

(72) Inventors: Julien BOUNOUAR, Annecy Cedex (FR); Stéphane MARCHAL, Annecy Cedex (FR); Olivier LE BARILLEC, Annecy Cedex (FR)

(73) Assignee: PFEIFFER VACUUM, Annecy (FR)

(21) Appl. No.: 19/108,541

(22) PCT Filed: Sep. 6, 2023
(86) PCT No.: PCT/EP2023/074475

§ 371 Date: Mar. 4, 2025

(30) Foreign Application Priority Data
Sep. 20, 2022 (FR) 2209476

(51) Int. Cl. G01N 33/00

(52) U.S. Cl. CPC G01N 33/0032; G01N 33/0016; G01N 33/0073

(57) ABSTRACT

A method measures airborne molecular contamination via a measuring station including at least one first and one second gas analysers, at least one sampling line, and at least one valve. The method includes measuring molecular contamination concentrations in the sampling line by the first gas analyser for a first predefined time, fluidic isolation between the first gas analyser and the sampling line via at least one valve, and measuring molecular contamination concentrations in the sampling line by the second gas analyser for a second predefined time.

### TECHNICAL FIELD

The present invention relates to a method for measuring airborne molecular contamination by means of a measuring station. The present invention also relates to this kind of station for measuring airborne molecular contamination, which may be intended in particular for surveillance of airborne molecular contamination concentrations in white rooms, such as white rooms of semiconductor manufacturing plants.

### TECHNICAL BACKGROUND

In the semiconductor manufacturing industry substrates, such as semiconductor wafers or photomasks, must be protected from airborne molecular contamination (AMC) in order for the latter not to damage the microchips or electronic circuits on the substrates. To this end the substrates are contained in atmospheric storage and transport boxes enabling transport of the substrates from one equipment to another or storage thereof between two manufacturing steps. Moreover, the transport boxes and the equipment are disposed in white rooms in which the level of particles is minimised and the temperature, relative humidity and pressure are maintained at precise levels.

In white rooms airborne gas species may have different sources and be of different kinds, for example acids, bases, condensable elements, doping elements. These molecules may originate in the air inside the semiconductor manufacturing plant or notably be released by semiconductor wafers that have undergone previous manufacturing operations.

Gas analysers in white rooms enable evaluation of the airborne gas species concentration at atmospheric pressure in real time, notably that of water vapour and a few acids. These gas analysers measuring the gaseous atmosphere around them, it is generally necessary to provide a gas analyser in each zone to be tested in the white room.

There is a need to increase the number of gas species measured and the number of test zones in order to reduce the risks of contamination of the substrates. However, multiplying the gas analysers in each zone and multiplying these zones to be tested quickly renders this solution very costly.

To reduce costs a measuring unit has been proposed combining different analysers. These analysers are chosen as a function of the gas chemistry and the nature of the gas species to be measured. The measuring unit has a plurality of inlet ports each addressing a particular test zone of the white room. The white rooms may be large and, the number of test zones also increasing, it may prove necessary to use a large number of sampling lines. The sampling lines enable air to be routed from the test zones to the gas analysers. The lengths of these lines often reach several tens of metres or even hundreds of metres.

One solution consists in aspirating the gases into the sampling lines. Measurement of the different gas chemistries can be effected as a function of the number of analysers present. The analysers being independent of one another, the aspiration flow may be different in the sampling lines.

However, some sampling lines may be very long, notably several hundred metres long, so a reduction of pressure may be observed at the inlet of the gas analysers. In fact, the sampling lines generally have a relatively small diameter, notably around 4 mm, acting as a restriction to the passage of the gas flow. The pressure reduction is also in part linked to the flow drained by the analysers.

One possible result of such pressure reduction is that one or more gas analysers, depending on their technology, no longer measure in their usable pressure range or pressure tolerance range around atmospheric pressure, which can generate falsified or unusable measurement results. This refers to gas analysers having a small pressure tolerance range, referred to as sensitive to pressure variations, notably such as those based on ion mobility spectrometry.

One solution could be to increase the diameter of the sampling lines in order to increase the conductance and thus to limit the resulting pressure reduction. However, this solution increases the surface areas of the lines liable to adsorb gases. These sampling lines may subsequently release some of the gas species conveyed, risking complication of the interpretation to be given to the measurement results. In order to limit this it is preferable to use a material of very high purity with a very smooth interior surface state to manufacture the sampling lines. This may in particular refer to a material enriched with fluorine, such as a fluoropolymer, for example perfluoroalkoxy (PFA) or polytetrafluoroethylene (PTFE).

However, increasing the diameter of each sampling line, when there may be up to one or two hundred lines, and over a length that can reach several hundred metres, using this kind of specific material considerably increases the overall cost of the measuring unit.

Moreover, the sampling lines are generally conditioned before a new measurement to eliminate the memory effect of the lines in some applications, notably when the gas species under surveillance are particularly adherent to the walls. The effect of increasing the diameter is to extend the degassing time and consequently the conditioning time for each sampling line.

Another solution could be to reduce the length of the sampling lines, for example to a few tens of metres, or to limit the number of gas analysers. However, solutions of this kind have only very limited benefit.

### SUMMARY OF THE INVENTION

One of the objects of the present invention is to propose a measuring station and method that solve at least partly one or more of the aforementioned disadvantages.

To this end the invention has for object a method for measuring airborne molecular contamination by means of a measuring station including at least one first gas analyser, at least one second gas analyser, at least one sampling line connected to an inlet of the first gas analyser and to an inlet of the second gas analyser, and at least one valve configured for selectively putting into fluidic communication or fluidic isolation the first gas analyser and the sampling line. Said method includes the following steps:
measurement of molecular contamination concentrations in the sampling line by the first gas analyser for a first predefined time,
fluidic isolation between the first gas analyser and the sampling line by means of the at least one valve, and
for a second predefined time, when the first gas analyser and the sampling line are fluidically isolated, measurement of molecular contamination concentrations in the sampling line by the second gas analyser.

This kind of method enables sequencing of the measurements. The first gas analysers may effect a measurement on one sampling line. The second gas analysers may effect their measurements when the first gas analysers have been fluidically isolated from the sampling line in order to limit any possible pressure reduction.

The invention also concerns a station for measuring airborne molecular contamination configured to use an airborne molecular contamination including at least one first gas analyser, at least one second gas analyser, at least one sampling line connected to an inlet of the first gas analyser and to an inlet of the second gas analyser, and at least one valve configured to enable selective putting into fluidic communication or fluidic isolation the first gas analyser and the sampling line.

Said station is advantageously configured to employ a method of measuring airborne molecular contamination as defined above.

The first gas analyser may be configured to measure at least one molecular contamination concentration in the sampling line for a first predefined time.

The at least one valve may be configured fluidically to isolate the first gas analyser and the sampling line.

The second gas analyser may be configured to measure at least one molecular contamination concentration in the sampling line for a second predefined time when the first gas analyser and the sampling line have been fluidically isolated.

Said method and/or said station may further have one or more of the following features described hereinafter, separately or in combination.

The sampling line may be in fluidic communication with the second gas analyser for the first predefined time and for the second predefined time.

Alternatively, the sampling line may be fluidically isolated from the second gas analyser for the first predefined time. The sampling line may be put into fluidic communication with the second gas analyser for the second predefined time by means of another valve.

The second gas analyser may be fluidically isolated from the sampling line after the second predefined time by means of the at least one valve.

In accordance with a first embodiment the first gas analyser may have a first pressure tolerance range.

The second gas analyser may have a second pressure tolerance range. The second pressure tolerance range may be narrower than said first range and included in said first range.

The first tolerance range may be around atmospheric pressure, notably centred on atmospheric pressure.

The second tolerance range may be around atmospheric pressure, notably centred on atmospheric pressure.

The first pressure tolerance range corresponds for example to atmospheric pressure +/−500 hPa, preferably +/−150 hPa.

The second pressure tolerance range corresponds for example to atmospheric pressure +/−70 hPa, preferably +/−30 hPa.

The first gas analyser can therefore effect a measurement on the sampling line, even if a large pressure reduction, for example greater than 30 mbar, is observed at the inlet of the gas analysers. The fluidic isolation between the first gas analysers and the sampling line makes it possible to ensure that each second gas analyser sees at its inlet a pressure that it is in its optimum pressure tolerance range.

The station may include at least two first analysers. For the first predefined time the first gas analysers can measure the molecular contamination concentrations simultaneously.

The station may include at least two second gas analysers.

Said method may include a step of measurement of molecular contamination concentrations by each second gas analyser, independently and successively.

After each measurement by one second gas analyser that second gas analyser may be fluidically isolated from the sampling line before the next measurement by another second gas analyser.

In accordance with another aspect said method may comprise at least one step of measurement of at least one pressure value at the inlet of the first gas analyser.

Said method may comprise at least one step of comparison of the pressure value measured at the inlet of the first gas analyser with the first pressure tolerance range.

If the measured pressure value is in the first pressure tolerance range said method may comprise at least one step of measurement of molecular contamination concentrations in the sampling line by the first gas analyser for the first predefined time and at least one step of collection of measurement results from the first gas analyser by a control unit.

After fluidic isolation between the first gas analyser and the sampling line by means of the at least one valve, said method may comprise at least one step of measurement of at least one pressure value at the inlet of the second gas analyser.

Said method may comprise at least one step of comparison of the pressure value measured at the inlet of the second gas analyser with the second pressure tolerance range.

If the pressure value measured at the inlet of the second gas analyser is in the second pressure tolerance range said method may comprise at least one step of measurement of molecular contamination concentrations in the sampling line by the second gas analyser for the second predefined time and at least one step of collection of measurement results from the second gas analyser by a control unit.

In accordance with a second embodiment the first gas analyser and the second gas analyser may have the same predefined pressure tolerance range.

This predefined tolerance range may be around atmospheric pressure, notably centred on atmospheric pressure. For example, the tolerance range may correspond to atmospheric pressure +/−70 hPa, preferably +/−30 hPa. In accordance with another example the tolerance range may correspond to atmospheric pressure +/−500 hPa, notably +/−150 hPa.

The first gas analyser and/or the second gas analyser may employ a technology chosen from laser spectroscopy, optical cavity spectroscopy, mass spectrometry, proton transfer reaction mass spectrometry, ion mobility spectrometry, an electrochemical technology, a colorimetric technology, fluorescence spectroscopy, flame ionisation detection, a chemiluminescence technology, a resistive technology.

During a step of measurement of respective molecular contamination concentrations the first gas analyser and/or the second gas analyser may measure at least one concentration of at least one gas species chosen from hydrofluoric acid, hydrochloric acid, ammonia, at least one volatile organic compound, at least one acid, at least one amine, at least one doping agent, sulphur dioxide, at least one sulphur-containing compound, ozone, nitrogen oxide, water vapour.

The at least one valve may a controllable valve.

The at least one valve may be an on/off valve, a three-port valve.

In accordance with one example at least one valve may be connected to the inlet of at least one gas analyser.

The valve at the inlet of the gas analyser may be external to the gas analyser.

Alternatively, at least one gas analyser may include an internal valve.

Said station may include a control unit.

The control unit may be configured to collect measurement results from the first gas analyser and the second gas analyser.

The control unit may be configured to control the at least one controllable valve.

Said station may include at least one pressure gauge.

The pressure gauge may be configured to measure at least one pressure value at the inlet of the first and/or second analyser.

Said station may include a comparator configured to receive and compare the measured pressure value to a predefined pressure tolerance range.

The first predefined time may be greater than the second predefined time.

The first predefined time may be less than 150 s, for example 120 s. Alternatively, the first time may be less than or equal to 30 min, for example between 3 min and 6 min inclusive.

The second predefined time may be less than 50 s, for example 30 s.

### BRIEF DESCRIPTION OF THE DRAWINGS

Other advantages and features of the invention will become more clearly apparent on reading the following description given by way of non-limiting and illustrative example and from the appended drawings, in which:

FIG. 1 represents a schematic view of one embodiment of a station for measurement of airborne molecular contamination.

FIG. 2 shows a table representing the fluidic communication as a function of time between gas analysers and a sampling line of the measurement station in accordance with one embodiment.

FIG. 3 shows a table representing fluidic communication as a function of time between gas analysers and a sampling line of the measurement station in accordance with another embodiment.

In these figures identical elements bear the same reference numbers.

The following embodiments are examples. Although the description refers to one or more embodiments, this does not necessarily mean that each reference concerns the same embodiment or that the features apply to only one embodiment. Single features of different embodiments may equally be combined or interchanged to provide other embodiments.

In the description some elements may be indexed, for example first element or second element. In this case this is merely indexing to differentiate and to designate similar but not identical elements. This indexing does not imply any priority of one element with respect to another and such designations may readily be interchanged without departing from the scope of the present invention. Nor does this indexing imply an order in time.

### DETAILED DESCRIPTION

### Measurement Station

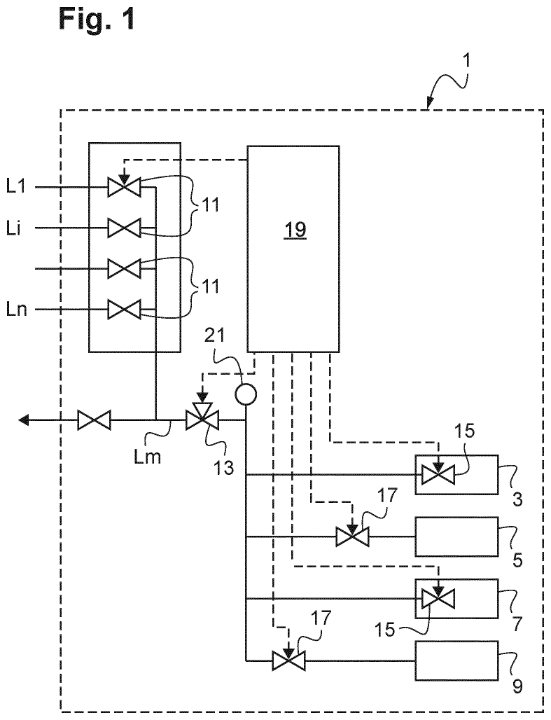

FIG. 1 shows one example of a station 1 for measurement of airborne molecular contamination. The measuring station 1 may be intended in particular for surveillance of molecular contamination concentrations in the atmosphere of white rooms, such as the white rooms of semiconductor manufacturing plants.

The measuring station 1 includes a predefined number of gas analysers 3, 5, 7, 9, one or more sampling lines L1, . . . , Li, . . . , Ln, and one of more valves 11, 13, 15, 17.

The sampling lines L1-Ln and/or the valves 11-17 may have internal surfaces intended to be in contact with the gases made of one or more materials limiting the adhesion of the gas species, such as one or more fluoropolymer materials, such as perfluoroalkoxy (PFA) or polytetrafluorethylene (PTFE).

In particular, the station 1 includes at least one first gas analyser 3, 7 and at least one second gas analyser 5, 9. Two first gas analysers 3, 7 and two second gas analysers 5, 9 are represented in the FIG. 1 illustrative example. Of course, these numbers are not limiting on the invention.

In operation, each gas analyser 3-9 may be put into fluidic communication with one sampling line L1-Ln at a time.

The first gas analyser or analysers 3, 7 is or are configured to measure at least one molecular contamination concentration in the associated sampling line Li with which it is or they are in fluidic communication.

The second gas analyser or analysers 5, 9 is or are configured to measure at least one molecular contamination concentration in the associated sampling line Li with which it is or they are in fluidic communication.

Each gas analyser 3, 5, 7, 9 enables measurement of the concentration of at least one gas species. The gas species measured is for example an acid, such as hydrofluoric acid (HF) or hydrochloric acid (HCl). In accordance with another example the gas species measured is a volatile organic compound (VOC) or ammonia (NH₃) or an amine. The gas species measured may also be sulphur dioxide (SO₂) or at least one sulphur-containing compound, or nitrogen oxide (NOₓ), ozone, water vapour, or at least one doping agent. The gas analyser 3, 5, 7, 9 may be adapted to measure one distinct gas species or a group of distinct gas species.

In accordance with one embodiment the measurement may be effected in real time, that is to say with a measurement time less than a few seconds, or even a few minutes, at low concentrations below one part per million (ppm) or one part per billion (ppb).

The gas analysers 3, 5, 7, 9 may include a respective internal pump for taking gas samples. Instead or in addition to this the measuring station 1 may include a pump (not represented) termed a sampling pump.

The gas analysers 3-9 are configured to sample a flow of air at around atmospheric pressure. The technology of the gas analysers 3-9 may necessitate internal operation under at least partial vacuum.

The pressure ranges of the gas analysers 3-9 are linked to their technologies. The gas analysers 3-9 are chosen according to the gas chemistry to be measured and advantageously other criteria such as response time, reliability and pressure tolerance range.

In accordance with a first embodiment the gas analysers 3-9 may be divided into at least two gas analyser groups or categories. The measuring station 1 includes for example a first group of gas analysers and a second group of gas analysers. Each group of gas analysers includes at least one gas analyser.

The groups of gas analysers are distinct. They may use different technologies.

The first group includes for example one or more gas analysers that are not very or not at all sensitive to a pressure variation or a pressure reduction relative to atmospheric pressure at the inlet of these gas analysers. To be more precise, if a pressure reduction is observed at the inlet of the gas analysers, for example if the sampling line in which the measurement is effected is very long, notably of the order of one or two hundred metres long, the pressure at the inlet of the gas analysers remains acceptable for operation thereof. These gas analysers of the first group remain in their pressure tolerance range even in the event of at least partial pressure reduction.

The second group may to the contrary include one or more gas analysers that are sensitive to a pressure variation or a pressure reduction relative to atmospheric pressure at the inlet of these analysers. The measurement results from such gas analysers may not be reliable if too great a pressure reduction is seen, for example greater than 30 mbar.

In accordance with the example depicted in FIG. 1 the first group includes the first gas analysers 3 and 7 and the second group includes the second gas analysers 5 and 9.

The first gas analyser or analysers 3, 7 may have a first pressure tolerance range. The first tolerance range may be around atmospheric pressure, notably centred on atmospheric pressure. The first pressure tolerance range corresponds for example to atmospheric pressure +/−500 hPa. To give a specific example, the first pressure tolerance range may correspond to atmospheric pressure +/−100 to 150 mbar, i.e. +/−100 to 150 hPa.

The different first gas analysers 3, 7 can simultaneously measure the molecular contamination concentrations in an associated sampling line Li.

The second gas analyser or analysers 5, 9 has or have a second pressure tolerance range narrower than the first range. This second range may notably be within the first range. The second tolerance range may be around atmospheric pressure, notably centred on atmospheric pressure. The second pressure tolerance range corresponds for example to atmospheric pressure +/−70 hPa. To give a specific example the second pressure tolerance range may correspond to atmospheric pressure +/−30 mbar, i.e. +/−30 hPa.

The second gas analyser or analysers 5, 9 may or may not be in fluidic communication with the sampling line Li during measurement by the first gas analysers 3, 7.

Each second gas analyser 5, 9 can measure molecular contamination concentrations independently of the other second gas analysers 5, 9. During these measurements the first gas analysers 3, 7 and the other second gas analysers 5, 9 are fluidically isolated from the sampling line Li.

By way of non-limiting example the first gas analyser or analysers 3, 7 may use a technology chosen from laser spectroscopy, optical cavity spectroscopy (cavity ring down spectroscopy (CRDS)), mass spectrometry, proton transfer reaction (PTR) mass spectrometry.

In a manner that is not limiting on the invention the second gas analyser or analysers 5, 9 may use a technology chosen from ion mobility spectrometry (IMS), an electrochemical technology, a colorimetric technology, fluorescence spectroscopy, in particular in the ultraviolet (UV) range, flame ionisation detection (FID), a chemiluminescence technology, a resistive technology.

By way of one particular non-limiting example a gas analyser using optical cavity spectroscopy (CRDS) may be adapted to measure hydrofluoric acid (HF) or hydrochloric acid (HCl) or ammonia (NH₃). In accordance with another example a gas analyser using ion mobility spectrometry (IMS) may be adapted to measure at least one acid or at least one amine. In accordance with a further example a gas analyser using UV fluorescence spectroscopy may be adapted to measure sulphur dioxide (SO₂). A gas analyser using flame ionisation detection (FID) or proton transfer reaction (PTR) mass spectrometry may be adapted to measure at least one volatile organic compound (VOC). In accordance with other variants a gas analyser using chemiluminescence may be adapted to measure nitrogen oxide (NOₓ), a gas analyser using a resistive technology may be adapted to measure water vapour, or a gas analyser using an electrochemical or colorimetric technology may be adapted to measure at least one doping element or doping agent.

In accordance with a second embodiment the gas analysers 3, 5, 7, 9 have the same or similar predefined pressure tolerance range(s). In other words, there is not necessarily any distinction in terms of pressure tolerance between the first gas analyser or analysers 3, 7 and the second analyser or analysers 5, 9.

The predefined tolerance range may be around atmospheric pressure. It is notably centred on atmospheric pressure. For example, the tolerance range corresponds to atmospheric pressure +/−70 hPa, preferably +/−30 hPa. In accordance with another example the tolerance range may correspond to atmospheric pressure +/−500 hPa, notably +/−150 hPa.

The first and second gas analysers 3-9 may use similar technologies or the same technology. This may notably be one of the technologies listed above in the description of the gas analysers in accordance with the first embodiment.

In one or other of the embodiments of the gas analysers 3-9 the sampling line or lines L1, . . . , Li, . . . , Ln is or are connected to an inlet of the first gas analyser or analysers 3, 7 and to an inlet of the second gas analyser or analysers 5, 9. The gas analysers 3-9 may therefore be put into fluidic communication with the sampling line or one of the sampling lines L1-Ln. A plurality of sampling lines L1-Ln may be connected to a common line Lm connected to the inlet of the analysers 3-9. The common line Lm may also be connected to an exhaust.

One end of each sampling line L1-Ln is intended to discharge into a test zone at ambient pressure, that is to say atmospheric pressure. The sampling lines L1-L4 connect the measuring station 1 to distinct test zones, for example at a distinct location in a white room. A plurality of sampling lines L1-Ln may discharge at distinct locations. The length of the sampling lines L1-Ln may vary between the different test zones to be connected and may be a few metres or a few tens of metres, for example more than 200 m.

The sampling pump if any may be fluidically connected to the common line connecting a plurality of sampling lines L1-Ln. The gas to be analysed by the gas analysers 3-9 can therefore be sampled in the sampling lines L1-Ln by such a pump or by the internal pump of the gas analysers 3-9.

In accordance with one example a valve 11 termed a sampling valve may be arranged on each sampling line L1-Ln. At least one other valve 13, also referred to as a sampling valve, may be arranged on the common line Lm if any connected to the sampling lines L1-Ln.

One or more of the valves 11-17 are arranged and configured to enable selective fluidic communication or fluidic isolation between at least one gas analyser 3-9 and the sampling line or one of the sampling lines L1-Ln.

To this end one or more valves 15, 17 termed measuring valves may be provided. One or more valves 15, 17 may be integrated into or connected to the inlet of one or more of the gas analysers 3-9. At least one valve 15, 17 may be associated with each gas analyser 3-9. If the gas analyser 3-9 includes the measuring valve 15 or 17, the latter is an internal valve. Alternatively the measuring valve 15 or 17 may be external to the gas analyser 3-9.

In particular, at least one measuring valve 15 may be integrated into or connected to the inlet of the first gas analyser or analysers 3, 7.

At least one second measuring valve 17 may be integrated into or connected to the inlet of the second gas analyser or analysers 5, 9.

In the particular example represented in FIG. 1 the first measuring valves 15 are internal valves of respective first gas analysers 3, 7. In this example the second measuring valves 17 are external to the second gas analysers 5, 9. Of course, this example is not limiting on the invention. The converse set up may be envisaged. It may equally be envisaged that all the gas analysers have internal valves or conversely that the valves are all external to the gas analysers.

One or more valves 11-17 may be controllable valves, for example solenoid valves or pneumatic valves. They may be on or off (open or closed) valves. Alternatively or additionally one or more valves may be three-port valves.

The measuring station 1 may further include a control unit 19.

The control unit 19 may be configured to collect measurement results from the first gas analysers 3, 7 and the second gas analysers 5, 9.

The control unit 19 may be connected to one or more controllable valves 11-17, notably to the measuring valves 15, 17. The control unit 19 is configured to control, for example to open or close, the valves, 15, 17 to enable fluidic communication or fluidic isolation between one or more gas analysers 3-9 and the sampling lines L1-Ln.

The control unit 19 may notably control sequencing of the measurements by controlling the valves 11, 13, 15, 17.

The gas analysers 3-9 are for example mounted in a frame of the measuring station 1 that may be connected to an electrical cabinet (not represented) carrying the control unit 19 for example.

The measuring station 1 may equally include at least one pressure gauge 21 configured to measure at least one pressure value at the inlet of the first gas analysers 3, 7 and/or the second gas analysers 5, 9. This is for example a differential pressure gauge 21.

The measuring station 1 may include a comparator (not represented) configured to receive and compare the measured pressure value to a predefined pressure tolerance range. There may be the same pressure tolerance range for all the gas analysers 3-9 or to the contrary distinct pressure tolerance ranges depending on the type of gas analyser.

The control unit 19 may optionally be configured to collect measurement results from the first gas analysers 3, 7 and/or the second gas analysers 5, 9 if the pressure value measured at the inlet of these gas analysers 3-9 is in the associated pressure tolerance range or to the contrary to ignore these measurement results if the measured pressure value is not in that pressure tolerance range.

### Measurement Method

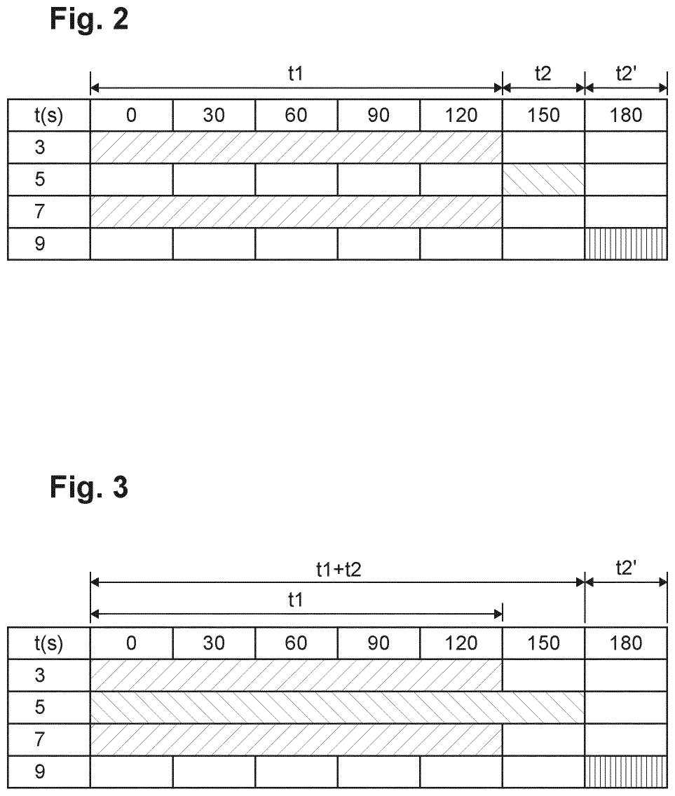

The measurement station 1 is advantageously configured to use a method for measuring airborne molecular contamination described hereinafter with reference to FIG. 1 and to the tables in FIGS. 2 and 3, representing putting the gas analysers 3-9 into fluidic communication with a sampling line as a function of the time t in seconds.

Generally speaking, in this measurement method the gas analysers 3-9 may successively or simultaneously sample a gas flow and carry out a measurement in a sampling line Li with which they are in fluidic communication. These measurements, and notably the taking into account thereof by the control unit 19, may be conditioned by a predefined time and/or a measured pressure value at the inlet of the gas analysers 3-9, as described in detail hereinafter.

On starting up the measurement method one or more gas analysers 3-9 may already be in fluidic communication with the sampling line or one of the sampling lines Li.

Alternatively, the method may include at least one preliminary step of putting into communication at least one first gas analyser 3, 7 and/or at least one second gas analyser 5, 9 and the sampling line Li. This putting into fluidic communication is effected by means of one or more valves, notably the so-called measuring valves 15 or 17. In the case of controllable valves the control unit 19 controls the valve or valves 15, 17, for example to the open position in the case of on-off control. The valves on the other sampling lines are for example closed.

The time of fluidic communication between the sampling line Li and the first gas analyser or analysers 3, 7 is symbolised by first hatching lines in the FIGS. 2 and 3 tables, toward the right in those figures. The time of fluidic communication between the sampling line Li and a second gas analyser 5 is symbolised by second hatching lines, toward the left in FIGS. 2 and 3. The time of fluidic communication between the sampling line Li and another second gas analyser 9 is symbolised by vertical hatching lines.

### First Embodiment

There are described hereinafter the steps of the method in accordance with a first embodiment with one or more so-called non-sensitive first gas analysers 3, 7 with a first pressure tolerance range and one or more so-called sensitive second gas analysers 5, 9 with a second narrower pressure tolerance range inside the first range.

The method includes at least one step of measuring molecular contamination concentrations in the sampling line Li by the first gas analyser or analysers 3, 7 for a first predefined time t1.

The first predefined time t1 may be less than 30 minutes, for example between 3 minutes and 6 minutes inclusive. In one particular instance the first predefined time may be less than 150 s, for example equal or substantial equal to 120 s. The first predefined time t1 is 120 s in the non-limiting illustrative example from FIG. 2 or 3. In accordance with a further example the first predefined time t1 may be less than 50 s, for example equal or substantially equal to 30 s.

If there are several of them, all the first gas analysers 3, 7 may be put into communication with a sampling line Li and simultaneously measure the molecular contamination concentrations for the first predefined time t1.

The measurements by the first gas analysers 3, 7 may be carried out whether the second gas analysers 5, 9 are in fluidic communication or not with the sampling line Li in which the measurement is carried out. The number of gas analysers 3-9 simultaneously sampling a gas sample on the sampling line Li on which the measurement is effected has no negative impact on the functioning of the first gas analysers 3, 7.

There may optionally be envisaged at least one step of measuring at least one pressure value at the inlet of the first gas analysers 3, 7. The pressure value measured at the inlet of the first gas analysers 3, 7 may be compared to the first predefined pressure tolerance range. If the measured pressure value is in that first pressure tolerance range measurement results from the first gas analysers 3, 7 may be collected, notably by the control unit 19. If not, those measurement results may be ignored by the control unit 19.

At the end of measurement by the first gas analysers 3, 7 i.e. after the first predefined time t1, the first gas analyser or analysers 3, 7 may be fluidically isolated from the sampling line Li, notably by means of one or more valves, in particular by the first so-called measuring valve or valves 15. The control unit 19 can control the first valve or valves 15, for example to close it or them.

The method may then include a step for putting into fluidic communication the second gas analyser 5, 9 and the sampling line Li, if it was previously isolated from the latter. This putting into fluidic communication may be effected by means of one or more valves, in particular by means of the second so-called measuring valve 17. The control unit 19 can control this second valve 17, for example to open it. On the other hand, if the second gas analyser 5, 9 was already in fluidic communication with the sampling line Li this step of putting into fluidic communication is not necessary.

### First Example

Moreover, one or more second gas analysers 5, 9 can be fluidically isolated from the sampling line Li for the first predefined time t1, as represented in the FIG. 2 example.

The method further includes at least one step during which a second gas analyser 5, 9 can measure molecular contamination concentrations in the sampling line Li for a second predefined time t2; t2′. This kind of step of measurement by the second gas analyser 5, 9 may be executed after fluidic isolation between the first gas analyser or analysers 3, 7 and the sampling line Li.

The second predefined time t2; t2′ may be different from the first predefined time t1. For example, the first predefined time t1 may be greater than the second predefined time t2; t2′. Alternatively, the second predefined time t2; t2′ may be similar to or equal to the first predefined time t1.

By way of specific example, the second predefined time t2; t2′ may be less than 50 s, for example equal or substantially equal to 30 s.

Each second gas analyser 5, 9 may be intended to carry out a measurement, individually or separated in time, if the first gas analysers 3, 7 or even all the other gas analysers are fluidically isolated from the sampling line Li. This makes it possible to limit the pressure reduction that may be observed at the inlet of the second gas analysers 5, 9.

The steps of measurement by the second gas analysers 5, 9 may be successive.

To this end the sampling line Li is put into fluidic communication with a second gas analyser 5 for a second predefined time t2 at the end of the first predefined time t1, i.e. from 120 s to 150 s in the FIG. 2 example. This putting into fluidic communication may be effected by means of the associated second valve 17 controlled for example by the control unit 19. The second gas analyser 5 then measures molecular contamination concentrations in the sampling line Li.

For this second predefined time t2 the sampling line Li is fluidically isolated from the other gas analysers 3, 7, 9. The control unit 19 can control the first valves 15 and where appropriate the second valve 17 associated with the other second gas analyser 9, for example to close it or them

The selected second gas analyser 5 is therefore the only one in fluidic communication with the sampling line Li, which makes it possible to prevent a large pressure reduction, with the result that the pressure at the inlet of the second gas analyser 5 is in the optimum pressure tolerance range of that second gas analyser 5, even if it is a gas analyser said to be sensitive to pressure variation or pressure reduction relative to atmospheric pressure.

There may equally be envisaged at least one step of measurement of at least one pressure value at the inlet of the second gas analyser 5. During a subsequent step the measured pressure value may be compared to the second pressure tolerance range. If the measured pressure value is in the second pressure tolerance range measurement results from the second gas analyser 5 may be collected, notably by the control unit 19, see also FIG. 1. If not, these measurement results may be ignored by the control unit 19.

After each measurement by a second gas analyser, for example the second gas analyser 5, the latter may be fluidically isolated from the sampling line Li before subsequent measurements by another second gas analyser 9. The fluidic isolation is obtained by means of one or more valves, in particular the second measuring valve 17 associated with the second gas analyser 5, which may be controlled by the control unit 19, for example to close it.

Another second gas analyser 9 can then be put into fluidic communication with the sampling line Li for a new predefined time t2′. This putting into fluidic communication may be effected by means of the second valve 17 associated with this other second gas analyser 9, controlled for example by the control unit 19.

A new measurement may be carried out for the new predefined time t2′. The new measurement is carried out independently of the preceding measurement and at a distinct time. In the example depicted the new measurement is carried out at the end of the preceding second predefined time t2 between 150 s and 180 s. The predefined time t2′ for this new measurement may be the same as the preceding second predefined time t2, in this example 30 s. This new predefined time t2′ is equally termed the second predefined time.

As previously the number of gas analysers in fluidic communication with the sampling line Li being limited, the pressure at the inlet of this other second gas analyser 9 is in its optimum pressure tolerance range.

As for the preceding second gas analyser 5, collection of measurement results from this other second gas analyser 9, notably by the control unit 19, may be conditioned by the pressure value measured at the inlet of this second gas analyser 9 and compared to the second pressure tolerance range.

At the end of the new measurement the measuring method may include a step for fluidically isolating this other second gas analyser 9 from the sampling line Li, notably by means of the corresponding second measuring valve 17.

If there are more than two second gas analysers the various steps of putting them into fluidic communication with the sampling line Li and of molecular contamination concentration measurement, and where applicable of comparison of a pressure value measured at the inlet of each second gas analyser to the second pressure tolerance range, then of fluidic isolation, may be repeated for each second gas analyser, independently and successively. The predefined time of measurement of molecular contamination concentrations may be the same for at least some or even all of the second gas analysers.

### Second Example

Referring to FIG. 3, another example differs from the first example described hereinabove in that the sampling line Li is also put into fluidic communication with at least one second gas analyser, the gas analyser 5 in the example depicted, for the first predefined time t1 during which the first gas analysers 3, 7 carry out their measurements.

The sampling line Li is therefore put into fluidic communication with the second gas analyser 5 for the first predefined time t1 and for the second predefined time t2, i.e. in the example depicted from 0 s to 150 s.

The second gas analyser 5 therefore takes a gas sample simultaneously with the first gas analysers 3, 7, which ensures effective conditioning of the second gas analyser 5.

At the end of the first measurement, in this example after 120 s, the sampling line Li is fluidically isolated from the first gas analysers 3, 7 as described above but remains in fluidic communication with the second gas analyser 5, which can therefore carry out and terminate the measurement for the second predefined time t2, from 120 s to 150 s in this example. This makes it possible to avoid too great a pressure reduction at the inlet of the second gas analyser 5. The latter having been conditioned previously by “seeing” the gas flow for the first predefined time t1, the measurement actually carried out for the second predefined period t2 is improved.

The following steps and other features of the first example apply to this second example and are not described again.

In the example represented in the FIG. 3 table one of the second gas analysers 9 is fluidically isolated from the sampling line Li for the first predefined time t1. In accordance with a variant that is not represented a plurality of or even all of the second gas analysers 5, 9 may be put into fluidic communication with the sampling line Li for the first predefined time t1. This enables the gas to stabilise in terms of adsorption/desorption in all the analysers. The reliability of the measurements from the second gas analysers conditioned in this way is improved.

### Second Embodiment

The method may be executed in accordance with a second embodiment when the first gas analyser or analysers 3, 7 and the second gas analyser or analysers 5, 9 have the same predefined pressure tolerance range. Only differences relative to the first embodiment are described hereinafter.

The first gas analyser or analysers 3, 7 may be put into communication with a sampling line Li and measure molecular contamination concentrations for the first predefined time t1. The second gas analyser or analysers 5, 9 may or may not be in fluidic communication with the sampling line Li for this first predefined time t1. The second gas analyser or analysers 5, 9 can then be put into communication with the sampling line Li and measure the molecular contamination concentrations for the second predefined time t2, t2′. The first gas analyser or analysers 3, 7 may or may not be in fluidic communication with the sampling line Li for this second predefined time t2′.

In particular, each gas analyser 3-9 may be intended to carry out individual measurements separated in time when the other gas analysers are fluidically isolated from the sampling line Li.

The steps of measurement by the different gas analysers 3-9 may be successive.

To this end the sampling line Li is put into fluidic communication with a gas analyser 3, 5, 7 or 9 for a predefined time. This putting into fluidic communication may be effected by means of the valve 15 or 17 connected to the inlet of this gas analyser, controlled for example by the control unit 19. For this predefined time the sampling line Li is fluidically isolated from the other gas analysers. The control unit 19 can control the valves 15 or 17 associated with the other gas analysers, for example to close them.

The selected gas analyser is therefore the only one in fluidic communication with the sampling line Li. After each measurement by a gas analyser 3-9 the latter may be fluidically isolated form the sampling line Li before the next measurement by another gas analyser 9.

The collection of measurement results from the gas analysers 3-9, notably by the control unit 19, may be conditioned by the pressure value measured at the inlet of each gas analyser 3-9 and compared to the pressure tolerance range, for example common to all the gas analysers 3-9.

The various steps of the measuring method according to either other embodiment may be repeated for each sampling line L1-Ln.

Moreover, the order of at least some of the steps of the measuring method may be reversed.

The gas analysers, notably when they are gas analysers 5, 9 sensitive to variation of pressure or pressure reduction at the inlet relative to atmospheric pressure may effect measurements one by one on a sampling line Li when the other gas analysers are fluidically isolated from the sampling line Li. The gas flow is weaker and the pressure reduction at the inlet of such gas analysers 5, 9 is greatly reduced. This enables the use of so-called pressure-sensitive gas analysers 5, 9.

When analysers 3, 7 said to be insensitive or little insensitive to variation of pressure or pressure reduction at the inlet relative to atmospheric pressure are provided, they may effect simultaneous measurement on the sampling line Li as other gas analysers equally sample or not a gas flow on that sampling line Li.

Finally, the so-called sensitive gas analysers 5, 9 may also sample a gas flow when the so-called insensitive gas analysers 3, 7 are carrying out their measurement before terminating their measurement when the latter are fluidically isolated from the sampling line Li, thus enabling effective conditioning.

1-16. (canceled)

17. A method for measuring airborne molecular contamination via a measuring station including at least one first gas analyser, at least one second gas analyser, at least one sampling line connected to an inlet of the first gas analyser and to an inlet of the second gas analyser, and at least one valve configured to selectively put into fluidic communication or fluidic isolation the first gas analyser and the sampling line, said method comprising:
measuring molecular contamination concentrations in the sampling line by the first gas analyser for a first predefined time;
fluidic isolating between the first gas analyser and the sampling line via the at least one valve; and
measuring, for a second predefined time, when the first gas analyser and the sampling line are fluidically isolated, the molecular contamination concentrations in the sampling line by the second gas analyser.

18. The method according to claim 17, in which the sampling line is put into fluidic communication with the second gas analyser for the first predefined time and the second predefined time.

19. The method according to claim 17, in which the sampling line is fluidically isolated from the second gas analyser for the first predefined time and put into fluidic communication with the second gas analyser for the second predefined time via another valve.

20. The method according to claim 17, in which:
the first gas analyser has a first pressure tolerance range, and
the second gas analyser has a second pressure tolerance range narrower than and included in said first range.

21. The method according to claim 20, in which:
the first pressure tolerance range corresponds to atmospheric pressure +/−500 hPa, and
the second pressure tolerance range corresponds to atmospheric pressure +/−70 hPa.

22. The method according to claim 20, in which:
the first pressure tolerance range corresponds to atmospheric pressure +/−150 hPa, and
the second pressure tolerance range corresponds to atmospheric pressure +/−30 hPa.

23. The method according to claim 20, in which the station includes at least two of the first gas analyser, and the at least two of the first gas analyser simultaneously measure molecular contamination concentrations for the first predefined time.

24. The method according to claim 20, in which:
the station includes at least two of the second gas analyser,
said method further comprises measuring molecular contamination concentrations via each of the at least two of the second gas analyser, independently and successively, and
after each measurement by one of the at least two of the second gas analyser, said second gas analyser is fluidically isolated from the sampling line before a next measurement by another second gas analyser.

25. The method according to claim 20, further comprising:
measuring at least one pressure value at the inlet of the first gas analyser,
comparing the pressure value measured at the inlet of the first gas analyser with the first pressure tolerance range, and
when the measured pressure value is in the first pressure tolerance range, measuring molecular contamination concentrations in the sampling line by the first gas analyser for the first predefined time and collecting measurement results from the first gas analyser via a control unit.

26. The method according to claim 20, in which after fluidic isolation between the first gas analyser and the sampling line via the at least one valve said method further comprises:
measuring at least one pressure value at the inlet of the second gas analyser,
comparing the pressure value measured at the inlet of the second gas analyser with the second pressure tolerance range, and
when the pressure value measured at the inlet of the second gas analyser is in the second pressure tolerance range, measuring molecular contamination concentrations in the sampling line by the second gas analyser for the second predefined time and collecting measurement results from the second gas analyser by a control unit.

27. The method according to claim 17, in which the first gas analyser and the second gas analyser have a same predefined pressure tolerance range.

28. The method according to claim 17, in which the first gas analyser and/or the second gas analyser uses a technology chosen from laser spectroscopy, optical cavity spectroscopy, mass spectrometry, proton transfer reaction mass spectrometry, ion mobility spectrometry, an electrochemical technology, a colorimetric technology, fluorescence spectroscopy, flame ionisation detection, a chemiluminescence technology, a resistive technology.

29. The method according to claim 17, in which, during a respective step of molecular contamination concentration measurement, the first gas analyser and/or the second gas analyser measures concentrations of at least one gas species chosen from hydrofluoric acid, hydrochloric acid, ammonia, at least one volatile organic compound, at least one acid, at least one amine, at least one doping agent, sulphur dioxide, at least one sulphur-containing compound, ozone, nitrogen oxide, water vapour.

30. A station for measuring airborne molecular contamination configured to use the method according to claim 17, said station comprising:
at least one first gas analyser;
at least one second gas analyser;
at least one sampling line connected to an inlet of the first gas analyser and to an inlet of the second gas analyser; and
at least one valve configured to enable selective putting into fluidic communication or fluidic isolation the first gas analyser and the sampling line, wherein:
the first gas analyser is configured to measure at least one molecular contamination concentration in the sampling line for a first predefined time,
the at least one valve is configured to isolate fluidically the first gas analyser and the sampling line, and
the second gas analyser is configured to measure at least one molecular contamination concentration in the sampling line for a second predefined time when the first gas analyser and the sampling line are fluidically isolated.

31. The station according to claim 30, in which the at least one valve is a controllable valve.

32. The station according to claim 30, further comprising a control unit configured to collect measurement results from the first gas analyser and from the second gas analyser.

33. The station according to claim 30, further comprising:
at least one pressure gauge configured to measure at least one pressure value at the inlet of the first gas analyser and/or the second gas analyser; and
a comparator configured to receive the measured pressure value and to compare the measured pressure value to a predefined pressure tolerance range.
